(19) United States
(12) Patent Application Publication
(10) Pub. No.: US 2009/0005362 A1
(43) Pub. Date: Jan. 1, 2009

(54) Compositions Comprising Antihistamines or Mast Cell Stabilizers, and Methods of Making and Using Same

(76) Inventors: Toan P. Vo, Pittsford, NY (US); Erning Xia, Penfield, NY (US)

Correspondence Address: Bausch & Lomb Incorporated, One Bausch & Lomb Place, Rochester, NY, 14604-2701, US

(21) Appl. No.: 11/768,602

(22) Filed: Jun. 26, 2007

(51) Int. Cl. A61K 31/335; A61K 31/352; A61K 31/4174; A61K 31/435; A61K 31/4465; A61K 31/451; A61K 31/55; A61P 27/14

(52) U.S. Cl. 514/217.05; 514/290; 514/291; 514/324; 514/330; 514/396; 514/450; 514/456

(57) ABSTRACT

Compositions comprise: (a) a material selected from the group consisting of antihistamines, stabilizers, salts thereof, and combinations thereof; and (b) a vasoconstrictor, decongestant, or a salt thereof, provided when the material consists of an antihistamine or a salt thereof, at least one antihistamine other than ketotifen and salts thereof is present. In certain embodiments, the compositions have a pH maintainable at about 5 or less during storage. The compositions can be used to treat, alleviate or ameliorate ocular allergic conditions.

### BACKGROUND

The present invention generally relates to compositions comprising antihistamines and/or mast cell stabilizers, and methods of making and using such compositions. In particular, the present invention relates to stable ophthalmic compositions containing antihistamines and/or mast cell stabilizers, and methods of making and using the same.

Ophthalmic compositions are useful for the treatment and temporary prevention of the signs and symptoms of ocular conditions, including allergic conjunctivitis, itching of the eye and redness of the eye. Methods of treating ocular conditions include administering to a human subject suffering therefrom or susceptible thereto an ophthalmic composition, for example, in the form of eye drops.

Ophthalmic compositions may also be useful for the treatment of dry eye condition, including inflammatory dry eye condition. Ophthalmic compositions may be formulated as single or multi dose units, with or without the use of a preservative, and may be manufactured by mixing various ingredients. The compositions may be packaged in single or multiple dosage forms, such as closed bottles, tubes, or other containers made from materials such as glass or plastic. In some cases, the packaging for the ophthalmic composition may be free or substantially free of antioxidant (e.g., as used in compositions described in U.S. Pat. Nos. 6,455,547 and 6,576,649).

Typically, the compositions are administered as drops, with one or more drops of the composition being applied to an eye of the subject suffering from or susceptible to ocular conditions one or more times per day, although the frequency of administration of such compositions may be dependent on multiple factors, including the makeup of the particular composition and the condition for which the compositions are used.

Ophthalmic solutions may contain buffers, various surfactants, stabilizers, isotonic agents and the like which aid in making the ophthalmic compositions more comfortable to the user. Oftentimes the ophthalmic solutions contain such agents and the like to maintain a predictable level of efficacy over a predetermined or expected lifetime.

Maintenance of efficacy and stability of ophthalmic solutions may be required to meet various federal health and safety regulations, e.g., shelf life testing, sterility, etc. For example, ophthalmic solutions may be required to contain expiration dates posted on their container, which may be predicated on the stability of the active ingredients and other conditions inherent in the formulation and environmental exposures of the product. Oftentimes stabilizing agents, although effective in maintaining specific properties of the formulation, are undesirable ingredients as they may cause adverse side effects in end-users or promote the degradation of active agents in the formulation.

Of particular importance for efficacy and commercialization of ophthalmic solutions is solution stability. Solution stability may be dependent on the interactions of all compounds present in the formulation as well as temperature and pH. Ophthalmic compositions typically have a pH anywhere from 4 to 6. The pH value is generally targeted to provide a specific level or range which provides the least amount of discomfort to the end user. Conventionally, a buffer (e.g., buffers including citrates, phosphates, borates, bicarbonates, sodium salts, potassium salts, etc. or a buffer with intrinsic antimicrobial properties such as a sodium borate/boric acid buffer) is used to achieve and maintain a desired pH of the compositions, and/or an acid or base is added to adjust the pH of the compositions to the desired level. However, certain otherwise pharmaceutically effective active agents may undergo degradation when formulated in the presence of buffering agents.

Furthermore, it may be desirable for an ophthalmic composition to include a plurality of active agents. In such situations, it may be difficult or uneconomical to meet a particular shelf life target or federal regulatory requirements due to some instability of the combination of the active agents or other interaction, e.g., with certain buffering agents. This may be the result of some chemical reactivity or incompatibility of the compounds or salts thereof, for example, which leads to degradation of one or more of the active agents. Such degradation shortens the shelf life of the solution and may render the formulation pharmaceutically ineffective or non-compliant with federal regulatory requirements.

Moreover, available ophthalmic solutions for treating or alleviating symptoms of ocular allergy often still lack one or more desirable characteristics, such as long-lasting efficacy for a particular condition or effectiveness in relieving more than one symptom.

Therefore, there is a continued need to provide improved ophthalmic compositions for treating, alleviating, or ameliorating an ocular allergic condition.

### SUMMARY

In general, the present invention provides a pharmaceutical composition comprising: (a) a material selected from the group consisting of mast cell stabilizers, antihistamines, salts thereof, and combinations thereof; and (b) a vasoconstrictor; provided when said material consists of an antihistamine or a salt thereof, at least one antihistamine other than ketotifen or a salt thereof is present.

In one aspect, the composition has a low initial pH.

In another aspect, the composition is a topical composition.

In still another aspect, the antihistamine is olopatadine or a salt thereof, or a combination of olopatadine and ketotifen or their salts.

In yet another aspect, the vasoconstrictor is naphazoline or a salt thereof.

In a further aspect, the present invention provides a method of preparing a stabilized pharmaceutical composition. The method comprises: (a) admixing a plurality of ingredients comprising: (1) a material selected from the group consisting of mast cell stabilizers, antihistamines, salts thereof, and combinations thereof; (2) a vasoconstrictor or a salt thereof; and (3) a carrier to form a mixture; and (b) adjusting a pH of said mixture to less than or equal to 5 with a pH adjusting material, thereby producing the composition; provided when said material consists of an antihistamine or a salt thereof, at least one antihistamine other than ketotifen or a salt thereof is present.

In still another aspect, said mast cell stabilizer, antihistamine, or a salt thereof in said composition is present in the composition in a concentration from about 0.01% to about 0.5% weight/volume (“w/v”); and said vasoconstrictor or a salt thereof is present in the composition in a concentration from about 0.01% to about 0.5% w/v. In one embodiment, said carrier comprises water.

In yet another aspect, the plurality of ingredients further comprises a tonicity-adjusting agent.

In a further aspect, the plurality of ingredients further comprises a buffering agent that is capable of maintaining the pH of the composition at less than or equal to about 5.

In a still another aspect, the method comprises adjusting the pH of the composition to a value between about 4.3 and 4.8.

In a further aspect, the method produces said composition, the pH of which is maintainable between about 4.3 to about 4.8 when said composition is kept at 40° C. and 20% relative humidity (“RH”) for at least 10 days.

In one embodiment, a method of preparing a stabilized ophthalmic composition is provided. The method comprises: (a) preparing a mixture comprising (1) a material selected from the group consisting of mast cell stabilizers, antihistamines, salts thereof, and combinations thereof in a concentration from about 0.01% to about 0.5% w/v; (2) a vasoconstrictor or a salt thereof in a concentration from about 0.01% to about 0.5% w/v; (3) glycerol in a concentration from about 2% to 6% w/v; and (4) water; and (b) adjusting a pH of the ophthalmic composition to a value in a range from about 4.3 to about 4.8 to provide said stabilized ophthalmic composition, wherein said pH of the ophthalmic composition is maintained in said range at 40° C. and 20% RH for at least 10 days; provided when said material consists of an antihistamine or a salt thereof, at least one antihistamine other than ketotifen or a salt thereof is present.

In yet another embodiment, a method of preparing a stabilized aqueous composition is provided. The method comprises admixing: (a) an aqueous composition comprising: (1) a material selected from the group consisting of mast cell stabilizers, antihistamines, salts thereof, and combinations thereof; and (2) a vasoconstrictor or a salt thereof, and (b) a pH adjusting agent to produce a mixture having a pH between 4.8 and 5; provided when said material consists of an antihistamine or a salt thereof, said antihistamine is other than ketotifen or a salt thereof, wherein the mixture is essentially free of buffering agents. The method further comprises allowing the pH of the mixture to adjust to between 4.3 and 4.8; thereby providing said stabilized aqueous composition such that no more than about 10% of said mast cell stabilizer, antihistamine, or salt thereof is degraded at 40° C. and 20% RH for at least 10 days.

### DETAILED DESCRIPTION

As used herein, unless otherwise specified, the concentration of a component or ingredient of a composition is represented by mass of the component or ingredient per total volume of the composition (i.e., g/mL), and is typically expressed as a percentage. For example, a concentration of 1% means 1 g per 100 mL of the composition.

As used herein, the term “active agent” or “active ingredient” refers to a compound or composition of matter that when administered to a subject (human or animal) causes a desired pharmacologic and/or physiologic effect by local and/or systemic action.

As used herein, the term “break point concentration” is defined generally as the concentration of a buffering agent that is insufficient to maintain the pH of a solution comprising one or more active agents at a temperature for a given time duration. By way of example, the break point concentration of a citrate buffer for a ketotifen salt solution is the concentration of citrate that allows a decrease in the pH value of the aqueous solution when kept at 40° C. and 20% RH for at least 10 days.

As used herein, the phrase “free or substantially free of buffer agent” refers to a composition absent a buffering agent or a composition where the amount of buffering agent is less than the break point concentration of the buffer.

In general, the present invention provides a pharmaceutical composition comprising: (a) a material selected from the group consisting of mast cell stabilizers, antihistamines, salts thereof, and combinations thereof; and (b) a vasoconstrictor or a salt thereof; provided when said material consists of an antihistamine or a salt thereof, at least one antihistamine other than ketotifen or a salt thereof is present; wherein the composition has a low initial pH. In one embodiment, said low initial pH is equal to or less than 5.

In one aspect, said mast cell stabilizers, antihistamines, vasoconstrictors, and salts thereof are capable of being used ophthalmically.

In another aspect, the composition is a topical composition.

In still another aspect, said mast cell stabilizer is selected from the group consisting of Cromolyn (also known as Cromoglycate), Nedocromil, salts thereof, and combinations thereof.

In yet another aspect, said antihistamine is selected from the group consisting of mepyramine, antazoline, diphenhydramine, carbinoxamine, doxylamine, clemastine, dimenhydrinate, pheniramine, chlorphenamine, dexchlorphenamine, brompheniramine, triprolidine, cyclizine, chlorcyclizine, hydroxyzine, meclizine, promethazine, alimemazine (trimeprazine), cyproheptadine, azatidine, acrivastine, astemizole, cetirizine, loratadine, mizolastine, azelastine, levocabastine, olopatadine, levocetirizine, desloratadine, fexofenadine, salts thereof, and combinations thereof. In one embodiment, said antihistamine is selected from the group consisting of acrivastine, astemizole, cetirizine, loratadine, mizolastine, azelastine, levocabastine, olopatadine, levocetirizine, desloratadine, fexofenadine, salts thereof, and combinations thereof. In another embodiment, said antihistamine comprises olopatadine.

In a further aspect, said vasoconstrictor is selected from the group consisting of tetrahydrozoline, ephedrine, oxymetazoline, phenylephrine, pseudoephedrine, tramazoline, xylometazoline, naphazoline, salts thereof, and combinations thereof.

In still another aspect, the material comprises olopatadine or a salt thereof.

In yet another aspect, the composition comprises: (a) olopatadine or a salt thereof; and (b) naphazoline or a salt thereof.

In a further aspect, the composition comprises: (a) a material selected from the group consisting of azelastine, levocabastine, olopatadine, salts thereof, and combinations thereof, (b) a mast cell stabilizer or salt thereof, and (c) a vasoconstrictor or a salt thereof. In one embodiment, said mast cell stabilizer comprises Cromolyn. In another embodiment, said mast cell stabilizer comprises Nedocromil. In still another embodiment, said vasoconstrictor is naphazoline.

In still another aspect, the composition comprises: (a) olopatadine or a salt thereof, (b) ketotifen or a salt thereof, (c) Cromolyn or salt thereof, and (d) a vasoconstrictor or a salt thereof.

In a further aspect, the present invention provides a method of preparing a stabilized pharmaceutical composition, wherein the stability of at least an active ingredient of the composition is maintained for an extended period of time. The method comprises: (a) admixing a plurality of ingredients comprising said at least an active ingredient and a carrier to form a mixture; and (b) adjusting a pH of said mixture to less than or equal to 5 with a pH adjusting material, thereby producing the composition having said stability.

In one embodiment, the stability of at least an active ingredient of the composition is maintained for at least 10 days after the manufacture of such composition. In another embodiment, such an extended period of time is at least one month. In still another embodiment, such an extended period of time is at least two, three, four, five, six, or twelve months, or longer.

In still another embodiment, the stability of the active ingredient is maintained when less than about 20% (or alternatively, in some embodiments with other active ingredients, less than 15%, or less than 10%, or less than 5%) (by weight) of the active ingredient has degraded or changed in such period of time.

In one aspect, a composition of the present invention is an aqueous solution.

In another aspect, a composition of the present invention is an oil-in-water emulsion.

In still another aspect, a composition of the present invention is administrable to an eye as a drop and becomes more viscous after contacting an ocular environment. In one embodiment, said composition comprises a carboxy-containing vinyl polymer that is capable of rendering said composition more viscous upon being applied to an ocular environment.

In yet another aspect, a composition of the present invention is a gel.

In a further aspect, the present invention provides a method of stabilizing an ophthalmic composition. The method comprises: (a) preparing a solution of at least an ophthalmic active agent and water; and (b) adjusting a pH of the ophthalmic composition to a value of 5 or lower. In one embodiment, the method provides for solution stability. In another embodiment, the method provides an ophthalmic composition that can provide comfort to a user of the composition.

In one embodiment said ophthalmic active agent is selected from the group consisting of mast cell stabilizers, antihistamines, slats thereof, and combinations thereof; provided when said material consists of an antihistamine or a salt thereof, at least one antihistamine other than ketotifen or a salt thereof is present.

The pH of an aqueous ophthalmic composition comprising an active agent, alone or in combination with other ingredients may be controlled when formulated with a buffer. However, merely achieving a stable pH of an ophthalmic composition comprising an active agent, at a predetermined value, may not be sufficient to maintain the stability of the active ingredient and/or ocular comfort of the ophthalmic composition. What is desirable is to provide an ophthalmic composition the pH of which does not exceed about 5 upon storage. Adding a buffer to the aforementioned composition to stabilize the pH of the composition may provide pH stability but may not provide a chemically stable solution. The presence of buffer agents may exacerbate the degradation and ocular discomfort of the composition.

In one aspect, a composition of the present invention is formulated such that an initial pH value thereof is equal to or less than about 5 without using a buffer. Such an initial pH can provide a degradation of an active ingredient of less than 10%.

A mast cell stabilizer, an antihistamine, or a salt thereof is present in a composition of the present invention in a concentration from about 0.001% to about 0.5%. Alternatively, said concentration is in the range from about 0.01% to about 0.5%, or from about 0.01% to about 0.3%, or from about 0.01% to about 0.2%, or from about 0.02% to about 0.1%, or from about 0.01% to about 0.05%, or from about 0.02% to about 0.04%. In one embodiment, a composition of the present invention comprises olopatadine in a concentration of about 0.2%. In another embodiment, a composition of the present invention comprises olopatadine in a concentration of about 0.1% and ketotifen fumarate in a concentration of 0.02%. In still another embodiment, a composition of the present invention comprises olopatadine in a concentration of about 0.2% and ketotifen fumarate in a concentration of 0.02%.

A vasoconstrictor or a salt thereof is present in a composition of the present invention in a concentration from about 0.001% to about 0.5%. Alternatively, said concentration is in the range from about 0.01% to about 0.5%, or from about 0.01% to about 0.2%, or from about 0.02% to about 0.1%, or from about 0.02% to about 0.05%, or from about 0.02% to about 0.04%. In some embodiments, the composition comprises naphazoline or a naphazoline salt in a concentration from about 0.02% to about 0.05%.

In one aspect, the method herein described provides stability to pharmaceutical compositions, such as ophthalmic solutions, adjusted with tonicity agents to approximate the osmotic pressure of normal lachrymal fluids, which, as stated in U.S. Pat. No. 6,274,626, is equivalent to a 2.5% solution of glycerol. Osmotic pressure, measured as osmolality, is generally about 225 to 400 mOsm/kg for conventional ophthalmic solutions.

However, in some embodiments, the pharmaceutical composition may be formulated to osmolality in the range from about 400 to about 875 mOsm/kg, for some desired purposes. In particular, such osmolality may be employed if the composition is formulated to be well tolerated by a user. For example, U.S. Patent Application No. 2006/0148899, incorporated herein by reference in its entirety, provides for ophthalmic solutions having osmolality from 400 to 875 mOsm/kg, which is said still to provide comfort to a user.

In another aspect, the tonicity agent (or tonicity-adjusting agent) comprises a nonionic tonicity agent. The nonionic tonicity agent is preferably glycerol, although other nonionic agents may be used such as, for example, urea, sorbitol, mannitol, propylene glycol, lactose, and dextrose. In certain embodiments, glycerol is used as the nonionic tonicity agent in a concentration from 2% to 6%, preferably from 3% to 5%, more preferably about 4% such that the composition has an osmolality from about 200 to about 700 mOsm/kg, preferably from about 220 to about 400 mOsm/kg, more preferably from about 220 to about 300 mOsm/kg. Ionic tonicity agents also can be used, such as sodium chloride or sodium sulfate. In certain embodiments, a combination of the tonicity agents may be used.

In still another aspect, ophthalmic compositions of the method further comprise a preservative. The ophthalmic compositions may optionally include a buffer agent to maintain the pH of the composition. In a preferred embodiment, the ophthalmic composition is free or substantially free of buffer agents that would have been routinely used to achieve and/or maintain the pH of pharmaceutical compositions.

In certain embodiments, an ophthalmic composition of the present invention further comprises a carboxy-containing vinyl polymer. In one embodiment, such a polymer comprises a lightly crosslinked carboxy-containing vinyl polymer.

Crosslinked carboxy-containing vinyl polymers used in practicing this invention are, in general, well known in the art. In one embodiment, such polymers may be prepared from at least about 90% (by weight) and, preferably, from about 95% to about 99.9% (by weight), based on the total weight of monomers present, of one or more carboxy-containing monoethylenically unsaturated monomers. Acrylic acid is the preferred carboxy-containing monoethylenically unsaturated monomer, but other unsaturated, polymerizable carboxy-containing monomers, such as methacrylic acid, ethacrylic acid, β-methylacrylic acid (crotonic acid), cis-α-methylcrotonic acid (angelic acid), trans-α-methylcrotonic acid (tiglic acid), α-butylcrotonic acid, α-phenylacrylic acid, α-benzylacrylic acid, α-cyclohexylacrylic acid, β-phenylacrylic acid (cinnamic acid), coumaric acid (o-hydroxycinnamic acid), umbellic acid (p-hydroxycoumaric acid), and the like can be used in addition to or instead of acrylic acid.

Such polymers may be crosslinked by a polyfunctional crosslinking agent, preferably a difunctional crosslinking agent. The amount of crosslinking should be sufficient to form insoluble polymer particles, but not so great as to unduly interfere with sustained release of the medicament. Typically, the polymers are only lightly crosslinked. Preferably, the crosslinking agent is contained in an amount of from about 0.01% to about 5% (by weight); more preferably, from about 0.1% to about 5% (by weight), and more preferably from about 0.2% to about 1% (by weight), based on the total weight of monomers present. Included among such crosslinking agents are non-polyalkenyl polyether difunctional crosslinking monomers such as divinyl glycol; 2,3-dihydroxyhexa-1,5-diene; 2,5-dimethyl-1,5-hexadiene; divinylbenzene; N,N-diallylacrylamide; N,N-diallymethacrylamide and the like. Also included are polyalkenyl polyether crosslinking agents containing two or more alkenyl ether groupings per molecule, preferably alkenyl ether groupings containing terminal CH₂═C< groups, prepared by etherifying a polyhydric alcohol containing at least four carbon atoms and at least three hydroxyl groups with an alkenyl halide such as allyl bromide or the like, e.g., polyallyl sucrose, polyallyl pentaerythritol, or the like; see, e.g., U.S. Pat. No. 2,798,053. Diolefinic non-hydrophilic macromeric crosslinking agents having molecular weights of from about 400 to about 8,000, such as insoluble di- and polyacrylates and methacrylates of diols and polyols, diisocyanatehydroxyalkyl acrylate or methacrylate reaction products of isocyanate terminated prepolymers derived from polyester diols, polyether diols or polysiloxane diols with hydroxyalkylmethacrylates, and the like, can also be used as the crosslinking agents; see, e.g., U.S. Pat. Nos. 4,192,827 and 4,136,250.

The crosslinked polymers may be made from a carboxy-containing monomer or monomers as the sole monoethylenically unsaturated monomer present, together with a crosslinking agent or agents. Preferably, the polymers are those in which up to about 40%; and more preferably, from about 0.0001% to about 20% by weight, of the carboxy-containing monoethylenically unsaturated monomer or monomers has been replaced by one or more non-carboxyl-containing monoethylenically unsaturated monomer or monomers containing only physiologically and ophthalmologically innocuous substituents, including acrylic and methacrylic acid esters such as methyl methacrylate, ethyl acrylate, butyl acrylate, 2-ethylhexylacrylate, octyl methacrylate, 2-hydroxyethyl-methacrylate, 3-hydroxypropylacrylate, and the like, vinyl acetate, N-vinylpyrrolidone, and the like; see U.S. Pat. No. 4,548,990 for a more extensive listing of such additional monoethylenically unsaturated monomers. Particularly preferred polymers are lightly crosslinked acrylic acid polymers wherein the crosslinking monomer is 2,3-dihydroxyhexa-1,5-diene or 2,3-dimethylhexa-1,5-diene. Preferred commercially available polymers include polycarbophil (Noveon AA-1) and Carbopol®.

The crosslinked polymers used in practicing this invention are preferably prepared by suspension or emulsion polymerizing the monomers, using conventional free radical polymerization catalysts, to a dry particle size of not more than about 50 μm in equivalent spherical diameter; e.g., to provide dry polymer particles ranging in size from about 1 to about 30 μm, and preferably from about 3 to about 20 μm, in equivalent spherical diameter. Using polymer particles that were obtained by mechanically milling larger polymer particles to this size is preferably avoided. In general, such polymers will have a molecular weight which has been variously reported as being from about 250,000 to about 4,000,000, and from 3,000,000,000 to 4,000,000,000.

In a preferred embodiment of the invention, the particles of crosslinked polymer are monodisperse, meaning that they have a particle size distribution such that at least 80% of the particles fall within a 10 μm band of major particle size distribution. More preferably, at least 90% and most preferably at least 95%, of the particles fall within a 10 μm band of major particle size distribution. Also, a monodisperse particle size means that there is no more than 20%, preferably no more than 10%, and most preferably no more than 5% particles of a size below 1 μm. The use of a monodispersion of particles will give maximum viscosity and an increased eye residence time of the ophthalmic medicament delivery system for a given particle size. Monodisperse particles having a particle size of 30 μm and below are most preferred. Good particle packing is aided by a narrow particle size distribution.

In one embodiment, the ophthalmic composition comprises a polymer component that consists essentially of one or more of the above-described crosslinked carboxy-containing polymers. This means that no additional polymers are present in the composition that would significantly affect the medicament release profile. Polymers and oligomers used as excipients, carriers, demulcents, or other non-medicament-interactive functions may still be included within the composition so long as the medicament release profile is not significantly altered. However, in this embodiment no polymer particles (water insoluble polymers) which materially affect release e.g., a cationic exchange resin) are present in addition to the crosslinked carboxy-containing polymers, and typically no other polymers (soluble or insoluble) of any kind are present in the composition.

When such crosslinked carboxy-containing vinyl polymer is present in an ophthalmic composition of the present invention, it is generally present in an amount ranging from 0.5 to 2%; preferably, from about 0.5% to about 1.2% (w/v); and more preferably, from about 0.6 to about 0.9% (w/v).

The ophthalmic compositions may include an acid or base to adjust the pH of the composition. The method is useful for the stabilization of ophthalmic solutions that have a pH value initially adjusted such that the pH value of the ophthalmic composition thereafter is maintainable between about 4.3 and about 5 (or, alternatively, between about 4.3 and about 4.8) at least for 10 days at 40° C. and 20% relative humidity. The solutions may be adjusted to any pH value such that the pH value of the ophthalmic composition thereafter is between about 4.3 and about 4.8. The solutions preferably may be initially adjusted to have a pH value above 4.5 or below 5. Preferably, the adjusted pH value is higher than the thereafter pH value of the composition. Most preferred is an initially adjusted solution having a pH value of about 4.8.

Typically, only small amounts of an acid or base will be needed to adjust the initial pH of the solution. By way of example, an acid and base suitable for adjusting the pH are hydrochloric acid and sodium hydroxide. Fumaric acid or fumaric acid/sodium fumarate may also be suitable to adjust the pH of the solution. A buffering agent (e.g., buffers including citrates, phosphates, borates, bicarbonates, sodium salts, potassium salts, etc.; or a buffer with intrinsic antimicrobial properties such as a sodium borate/boric acid buffer) may be used provided that the breakpoint buffer concentration is not exceeded. If a buffering agent is used, it is further preferred that no more than 10% of the concentration of any active agents in the composition is degraded, for example, at 40° C. and 20% RH for at least 10 days. Preferably, the ophthalmic solution is free or substantially free of buffering agent.

In one embodiment, the method is useful for the stabilization of compositions that include a preservative. In another embodiment, the method is useful for the stabilization of compositions that does not include a preservative. A preservative is preferred when the composition is packaged for multidose units, but may be absent from the composition if desired (e.g., in single dose units of the composition). Any preservative may be used with the compositions. Preservatives that may be used include Polyquad preservative (Alcon); perborate (e.g., sodium perborate from Ciba); Purite preservative (stabilized chlorine dioxide) (Allergan); other quaternary ammonium compounds such as benzalkonium chloride; alkyl-mercury salts of thiosalicylic acid such as, for example, thiomersal, phenylmercuric nitrate, phenylmercuric acetate, and phenylmercuric borate; parabens such as, for example, methylparaben or propylparaben; alcohols such as, for example, chlorobutanol, benzyl alcohol, and phenyl ethanol; guanidine derivatives such as, for example, chlorhexidine or polyhexamethylene biguanide; and the like. When a preservative is used in the composition, the preservative is typically provided in a concentration of about 0.005% to 0.02%, preferably 0.01%, although other concentrations may be used.

In one aspect, the method herein described provides for preparing a stabilized ophthalmic composition. The method comprises the steps of preparing an aqueous solution that comprises: (a) a material selected from the group consisting of mast cell stabilizers, antihistamines, salts thereof, and combinations thereof; and (b) a vasoconstrictor or a salt thereof; provided when said material consists of an antihistamine or a salt thereof, at least one antihistamine other than ketotifen or a salt thereof is present; wherein the composition has a low initial pH. In one embodiment, said low initial pH is equal to or less than 5.

In one embodiment, said solution comprises said material in a concentration from about 0.01% to about 0.5%; said vasoconstrictor or a salt thereof is present in a concentration from about 0.01% to about 0.1%; glycerol; benzalkonium chloride; and water. In another embodiment, said composition comprises olopatadine or a salt thereof in a concentration from about 0.1% to about 0.4%; naphazoline or a naphazoline salt is present in a concentration from about 0.01% to about 0.1%; glycerol; benzalkonium chloride; water; and a buffering agent. In still another embodiment, said composition comprises olopatadine or a salt thereof in a concentration from about 0.1% to about 0.4%; ketotifen or a salt thereof in a concentration from about 0.01% to about 0.04%; naphazoline or a naphazoline salt is present in a concentration from about 0.01% to about 0.1%; glycerol; and benzalkonium chloride. The method provides for adjusting the pH value of the aqueous solution to less than or equal to about 5 by adding a pH adjusting agent, and providing a stabilized ophthalmic composition where the pH value of the ophthalmic composition is maintainable between about 4.3 to about 4.8 when kept at 40° C. and 20% RH for at least 10 days.

As used herein, “maintainable” and grammatical equivalents thereof refers generally to a value of a property of a composition that is capable of being determined, that stays within a defined range or meets a specified target value during an interval of time associated with the storage of the composition. By way of example, an ophthalmic composition stored at 40° C. and 20% RH for at least 10 days that is determined to have a value corresponding to a defined range or specified target value, that value would be “maintainable.” An example of values that may be maintainable in accordance with the method herein described includes, without limitation, pH, ocular comfort and concentrations of the portion of an active agent that has not degraded or changed.

Concentrations of one or more active agents may change during storage. As used herein, “during storage” refers to any interval of time associated with the preparation, handling, sterilizing, transporting and distributing or marketing of the composition. The composition may be in whatever container or form as may be desirable. During storage also includes accelerated aging testing, or other testing as may be required by state and federal regulation, e.g., Food and Drug Administration (“FDA”) rules, regulations and protocols. By way of example, during storage includes 40° C. and 20% relative humidity for at least 10 days.

If the amount of active agent falls below a predetermined level, the composition may not provide the desired pharmaceutical effect that was intended. Furthermore, a shelf life of an ophthalmic composition may be correlated to or predicted by the amount of initial concentration of active agent(s) remaining after any given interval of time after formulating, packaging, sterilizing, etc. In one embodiment of a method of stabilizing an ophthalmic composition, a ratio of a determined concentration of an active agent in the composition after an interval of time from when the composition is formulated to an initial concentration of the active agent in the composition is provided. Generally the ratio may be expressed in percentage that has degraded. For example, an active agent with an initial concentration of 10 μg/L that degrades, for example, during storage to 8 μg/L of active agent would have 20% of the active agent degraded. Concentration of an active agent in a composition may be determined by a HPLC method. The initial concentration may correspond to a stated concentration of active agent on a label affixed to the container, box or insert provided with the ophthalmic composition, e.g, “label claim,” or to a pharmaceutically effective concentration of active agent.

Degradation of active agent refers generally to an active agent that has changed chemically such that a pharmaceutical property of the active agent is reduced or eliminated. Methods of determining the amount of degradation of active agents and concentrations of initial active agent remaining after an interval of time has elapsed are generally known. For example, an active agent that is detectable by a detection method generally used to determine a concentration of the active agent may be used to determine whether the concentration of the active agent has decreased relative to its initial formulated concentration. The detection method may only measure the concentration of active ingredient.

By adjusting the pH value of an ophthalmic solution such that the pH value of the ophthalmic composition is maintainable between about 4.3 and about 5 (or, alternatively, between about 4.3 and about 4.8) for example, at 40° C. and 20% RH for at least 10 days, it may be possible to substantially eliminate the need for a buffer agent, or it may provide for the use of very low concentrations of buffering agents. Providing ophthalmic compositions free or essentially free of buffer improves ocular comfort of the composition for the user. The method herein described may be useful for providing acceptable ocular comfort ophthalmic compositions comprising a mast cell stabilizer or an antihistamine, as well as compositions comprising a mast cell stabilizer or an antihistamine in combination with anti-redness agents, for example, naphazoline or naphazoline salts.

As used herein, ocular comfort refers to an effect of an ophthalmic composition on a user upon contact of the composition with an ocular space of the user. Ocular comfort is determined by a user responding to the introduction of one or more drops of a composition into the eye of the user. By way of example, the response may be graded on a numerical scale, from 1 to 10, 1 representing mostly discomfort, and 10 representing mostly comfort or the response may be an indication that the ocular comfort is acceptable or unacceptable.

The methods herein disclosed may be useful for the stabilization of an ophthalmic composition comprising, consisting of, or consisting essentially of: (a) a material selected from the group consisting of mast cell stabilizers, antihistamines, salts thereof, and combinations thereof; (b) a vasoconstrictor or a salt thereof; (c) a nonionic tonicity agent; and (d) water free or substantially free of buffer agents. In one embodiment, said material consists or consists essentially of olopatadine or a salt thereof, and ketotifen or a salt thereof. The nonionic tonicity agent may be present in a concentration such that the composition has an osmolality from 200 to 700 mOsm/kg, preferably from 200 to 400 mOsm/kg, more preferably, from about 220 to about 300 mOsm/kg. The nonionic tonicity agent may be glycerol. The concentration of glycerol may be about 2% to about 6%. The concentration of glycerol preferably may be from about 2% to about 3%, and more preferably, from about 2% to about 2.5%.

The methods herein disclosed may be useful for the stabilization of an ophthalmic composition comprising, consisting of, or consisting essentially of: (a) a material selected from the group consisting of mast cell stabilizers, antihistamines, salts thereof, and combinations thereof; (b) a vasoconstrictor or a salt thereof; and (c) a carrier; such that no more than about 10% of the mast cell stabilizers, antihistamines, or salts thereof is degraded at 40° C. and 20% RH for at least 10 days. The methods herein disclosed also may be useful for the stabilization of an ophthalmic composition comprising olopatadine or a salt thereof, and naphazoline or naphazoline salt, such that no more than about 5% of the naphazoline or the naphazoline salt is degraded at 40° C. and 20% RH for at least 10 days. Further, the methods herein disclosed may be useful for the stabilization of an ophthalmic composition of ketotifen and naphazoline (or their salts) such that no more than about 10% of the ketotifen or the ketotifen salt and no more than about 5% of the naphazoline or the naphazoline salt when combined together are degraded at 40° C. and 20% relative humidity for at least 10 days.

### EXAMPLES

The following examples are illustrative of the embodiments of the present invention and are not to be interpreted as limiting or restrictive. It should be understood that any numerical value, when measured, inherently contains certain uncertainties, as can be expressed by the standard deviation found in its respective measurements (e.g., pH), where such standard deviation can be determined or estimated. By way of example, a pH value is to be regarded as to be within a range of ±0.2.

In one example, a method of stabilizing an ophthalmic composition is provided as follows. The method comprises preparing a solution comprising: (a) a material selected from the group consisting of mast cell stabilizers, antihistamines, salts thereof, and combinations thereof in a concentration from about 0.01% to about 0.5%; (b) a vasoconstrictor or a salt thereof in a concentration from about 0.01% to about 0.1%; (c) glycerol in a concentration such that the solution has an osmolality of from 200 to 700 mOsm/kg (milliosmole/kg; and (d) and water; provided when said material consists of an antihistamine or a salt thereof, at least one antihistamine other than ketotifen or a salt thereof is present. A preservative may be added. The concentration of preservative may be from about 0.01% to about 0.03%, however, lower or higher concentrations may be used, in appropriate cases. The preservative can be benzalkonium chloride. The solution is prepared by contacting the ingredients with the water.

In another example, a method of preparing a stabilized ophthalmic composition is provided as follows. The method comprises preparing a composition consisting essentially of: (a) a material selected from the group consisting of mast cell stabilizers, antihistamines, salts thereof, and combinations thereof in a concentration from about 0.01% to about 0.5%; (b) a vasoconstrictor or a salt thereof in a concentration from about 0.01% to about 0.1%; (c) glycerol in a concentration such that the solution has an osmolality of from 200 to 700 mOsm/kg (milliosmole/kg; (d) benzalkonium chloride in a concentration of about 0.01%; (e) a buffering agent; and (f) water. The method further comprises adjusting a pH value of the composition to less than or equal to about 5. By adjusting the pH of the ophthalmic composition, the pH value is maintainable between about 4.3 and about 4.8 at 40° C. and 20% RH for at least 10 days.

In still another example, a method of stabilizing an ophthalmic composition is provided. The method comprises preparing a composition comprising: (a) olopatadine ({(11Z)-11-[3-(dimethylamino)propylidene]-6,11-dihydrodibenzo[b,e]oxepin-2-yl}acetic acid) or a salt thereof in a concentration from about 0.1% to about 0.3%; (b) a naphazoline base in a concentration from about 0.02% to about 0.05%; (c) glycerol in a concentration from about 2% to 2.5%; (d) and water. The pH value of the composition is adjusted, where the pH value is maintainable between about 4.3 and about 4.8 at 40° C. and 20% RH for at least 10 days. The osmolality of the composition is from about 200 to about 300 mOsm/kg. The composition may further comprise a citrate buffer in a concentration of about 0.002M or less. The composition may further comprise benzalkonium chloride in a concentration from about 0.01% to about 0.03%.

In yet another example, a method of stabilizing an ophthalmic composition is provided. The method comprises adjusting a pH value of the ophthalmic composition to less than about 5, the composition comprising: (a) a material selected from the group consisting of mast cell stabilizers, antihistamines, salts thereof, and combinations thereof; (b) a vasoconstrictor or a salt thereof; and (c) water, wherein the material is degraded less than 10% at 40° C. and 20% RH for at least 10 days. In one embodiment, the material comprises or consists of olopatadine or a salt thereof. In another embodiment, the material comprises or consists of olopatadine or a salt thereof, and ketotifen or a ketotifen salt. The material may be present in a concentration from about 0.01% to about 0.5%. The vasoconstrictor may be a naphazoline salt, preferably naphazoline hydrochloride. The naphazoline hydrochloride may be present in a concentration from about 0.01% to about 0.1%. In the above example, glycerol may be present, preferably in a concentration from about 2% to about 3% (or from about 2.1% to about 2.5%). The composition may further comprise a citrate buffer in a concentration such that no more than about 10% of said material degrades after at least 10 days at 40° C. and 20% RH. The composition may further comprise benzalkonium chloride in a concentration of about 0.005% to about 0.02%.

In one aspect, degradation analysis of the active ingredients in the formulations can be performed using HPLC using control samples for the active ingredients. Detection of the active ingredients may be achieved with a variable wavelength ultraviolet detector, as is known by people skilled in the art.

Non-limiting examples of compositions of the present invention are presented below.

### Example 1

| Ingredient | Concentration (%) |
|---|---|
| Olopatadine | 0.2 |
| Naphazoline HCl | 0.05 |
| Glycerol | 2.25 |
| HCl or NaOH | for pH adjustment to 4.8 |
| Water | q.s. 100 |

### Example 2

| Ingredient | Concentration (%) |
|---|---|
| Olopatadine | 0.2 |
| Naphazoline HCl | 0.05 |
| Glycerol | 2.25 |
| HCl or NaOH | for pH adjustment to 4.8 |
| Benzalkonium chloride | 0.01 |
| Water | q.s. 100 |

### Example 3

| Ingredient | Concentration (%) |
|---|---|
| Olopatadine | 0.1 |
| Ketotifen fumarate | 0.025 |
| Naphazoline HCl | 0.05 |
| Glycerol | 2.25 |
| HCl or NaOH | for pH adjustment to 4.5 |
| Water | q.s. 100 |

### Example 4

| Ingredient | Concentration (%) |
|---|---|
| Azelastine | 0.1 |
| Ketotifen fumarate | 0.025 |
| Ephedrine | 0.05 |
| Sodium chloride | 0.9 |
| HCl or NaOH | for pH adjustment to 4.5 |
| Water | q.s. 100 |

### Example 5

| Ingredient | Concentration (%) |
|---|---|
| Olopatadine | 0.1 |
| Ketotifen fumarate | 0.035 |
| Cromolyn | 0.1 |
| Naphazoline HCl | 0.05 |
| Glycerol | 2.25 |
| HCl or NaOH | for pH adjustment to 4.3 |
| Urea hydrogen peroxide | 0.01 |
| Water | q.s. 100 |

### Example 6

| Ingredient | Concentration (%) |
|---|---|
| Levocabastine | 0.15 |
| Ketotifen fumarate | 0.01 |
| Nedocromil | 0.15 |
| Oxymetazoline | 0.05 |
| Glycerol | 2.25 |
| HCl or NaOH | for pH adjustment to 4.3 |
| Benzalkonium chloride | 0.01 |
| Water | q.s. 100 |

### Example 7

| Ingredient | Concentration (%) |
|---|---|
| Olopatadine | 0.1 |
| Azatidine | 0.03 |
| Cromolyn | 0.15 |
| Naphazoline HCl | 0.05 |
| Sodium chloride | 0.9 |
| HCl or NaOH | for pH adjustment to 4.5 |
| Benzyl alcohol | 0.01 |
| Water | q.s. 100 |

### Example 8

| Ingredient | Concentration (%) |
|---|---|
| Olopatadine | 0.1 |
| Ketotifen fumarate | 0.03 |
| Cromolyn | 0.1 |
| Naphazoline HCl | 0.05 |
| Sodium chloride | 0.9 |
| Citrate buffer | q.s. for maintaining pH of about 4.8 |
| Benzyl alcohol | 0.01 |
| Water | q.s. 100 |

In certain other embodiments, a pharmaceutical composition consists essentially of: (a) a material selected from the group consisting of antihistamines, mast cell stabilizers, salts thereof, and combinations thereof; (b) a vasoconstrictor or decongestant, or a salt thereof; and (c) a pharmaceutically acceptable carrier; wherein each of said antihistamines, mast cell stabilizers, vasoconstrictor or decongestant, or salts thereof, when present, is present at a concentration from about 0.001% to about 0.2% (or alternatively, from about 0.001 to about 0.1%, or from about 0.005 to about 0.1%, or from about 0.05 to about 0.05%), and a pH of the composition remains at less than 5 at 40° C. and 20% RH for at least 10 days; provided when said material consists of an antihistamine or a salt thereof, at least one antihistamine other than ketotifen or a salt thereof is present.

In still certain other embodiments, a pharmaceutical composition consists essentially of: (a) a material selected from the group consisting of antihistamines, mast cell stabilizers, salts thereof, and combinations thereof; (b) a vasoconstrictor or decongestant, or a salt thereof; (c) a tonicity-adjusting agent; and (d) a pharmaceutically acceptable carrier; wherein each of said antihistamines, mast cell stabilizers, vasoconstrictor or decongestant, or salts thereof, when present, is present at a concentration from about 0.001% to about 0.5% (or alternatively, from about 0.001 to about 0.3%, or from about 0.005 to about 0.2%, or from about 0.05 to about 0.1%), and a pH of the composition remains at less than 5 at 40° C. and 20% RH for at least 10 days; provided when said material consists of an antihistamine or a salt thereof, at least one antihistamine other than ketotifen or a salt thereof is present. In certain aspects, the tonicity-adjusting agent is present at a concentration such that the osmolality of the composition is in the range from about 200 to about 700 mOsm/kg (or alternatively, from about 220 to about 600 mOsm/kg, or from about 250 to about 400 mOsm/kg, or from about 220 to about 350 mOsm/kg).

In still certain other embodiments, a pharmaceutical composition consists essentially of: (a) a material selected from the group consisting of antihistamines, mast cell stabilizers, salts thereof, and combinations thereof; (b) a vasoconstrictor or decongestant, or a salt thereof; (c) a tonicity-adjusting agent; (d) a preservative; and (e) a pharmaceutically acceptable carrier; wherein each of said antihistamines, mast cell stabilizers, vasoconstrictor or decongestant, or salts thereof, when present, is present at a concentration from about 0.001% to about 0.5% (or alternatively, from about 0.001 to about 0.3%, or from about 0.005 to about 0.2%, or from about 0.05 to about 0.1%), and a pH of the composition remains at less than 5 at 40° C. and 20% RH for at least 10 days; provided when said material consists of an antihistamine or a salt thereof, at least one antihistamine other than ketotifen or a salt thereof is present.

In still certain other embodiments, a pharmaceutical composition consists essentially of: (a) a material selected from the group consisting of antihistamines, mast cell stabilizers, salts thereof, and combinations thereof; (b) a vasoconstrictor or decongestant, or a salt thereof; (c) a tonicity-adjusting agent; (d) a preservative; (e) a viscosity-modifying agent; and (f) a pharmaceutically acceptable carrier; wherein each of said antihistamines, mast cell stabilizers, vasoconstrictor or decongestant, or salts thereof, when present, is present at a concentration from about 0.001% to about 0.5% (or alternatively, from about 0.001 to about 0.3%, or from about 0.005 to about 0.2%, or from about 0.05 to about 0.1%), and a pH of the composition remains at less than 5 at 40° C. and 20% RH for at least 10 days; provided when said material consists of an antihistamine or a salt thereof, at least one antihistamine other than ketotifen or a salt thereof is present.

In yet certain other embodiments, a pharmaceutical composition comprises: (a) a material selected from the group consisting of antihistamines other than ketotifen or salts thereof, mast cell stabilizers or salts thereof, and combinations thereof; (b) a vasoconstrictor or decongestant, or a salt thereof;; and (c) a pharmaceutically acceptable carrier; wherein each of said antihistamines, mast cell stabilizers, vasoconstrictor or decongestant, or salts thereof, when present, is present at a concentration from about 0.001% to about 0.5% (or alternatively, from about 0.001 to about 0.3%, or from about 0.005 to about 0.2%, or from about 0.05 to about 0.1%), and a pH of the composition remains at less than 5 at 40° C. and 20% RH for at least 10 days.

In still certain other embodiments, a pharmaceutical composition consists essentially of: (a) a material selected from the group consisting of antihistamines other than ketotifen and salts thereof, mast cell stabilizers or salts thereof, and combinations thereof; (b) a vasoconstrictor or decongestant, or a salt thereof; (c) a tonicity-adjusting agent; (d) a preservative; (e) a viscosity-modifying agent; and (f) a pharmaceutically acceptable carrier; wherein each of said antihistamines, mast cell stabilizers, vasoconstrictor or decongestant, or salts thereof, when present, is present at a concentration from about 0.001% to about 0.5% (or alternatively, from about 0.001 to about 0.3%, or from about 0.005 to about 0.2%, or from about 0.05 to about 0.1%), and a pH of the composition remains at less than 5 at 40° C. and 20% RH for at least 10 days.

In still certain other embodiments, at least 90% of each of said antihistamine drug, mast cell stabilizer drug, vasoconstrictor, or decongestant, when present, remains in the composition after storage at 40° C. and 20% RH for at least 10 days.

In still certain other embodiments, the vasoconstrictor or decongestant is selected from the group consisting of phenylephrine, oxymetazoline, xylometazoline, and combinations thereof.

In yet certain other embodiments, the antihistamine is selected from the group consisting of promethazine, alimemazine (trimeprazine), cyproheptadine, azatadine, azelastine, levocabastine, olopatadine, salts thereof, and combinations thereof.

In further embodiments, the composition is an aqueous solution, an oil-in-water emulsion, a dispersion, a gel, or a gelable formulation.

In still further embodiments, the composition has a viscosity in a range from about 5 to about 10000 mPa.s (or centipoises). Alternatively, the viscosity is in a range from about 5 to about 1000 mPa.s.

A composition of the present invention can be used to treat, ameliorate, or reduce a condition resulting from allergy. For example, a composition of the present invention can be applied topically to treat, ameliorate, or reduce the severity of allergic conjunctivitis or symptoms thereof, such as pink eye, itchy eye, or combinations thereof. A composition of the present invention may be applied to the ocular surface in the form of eye drops, in one or more drops once per day, twice per day, or three or more times per day.

In another embodiment, a composition of the present invention can be formulated to be used topically for dermatological applications to treat, ameliorate, or reduce allergic symptoms.

As used herein, “comprising,” “including,” “containing,” “characterized by,” and grammatical equivalents thereof are inclusive or open-ended terms that do not exclude additional, unrecited elements or method steps. “Comprising” is to be interpreted as including the more restrictive terms “consisting of” and “consisting essentially of.” As used herein, “consisting of” and grammatical equivalents thereof exclude any element, step, or ingredient not specified in the claim.

As used herein, “consisting essentially of” and grammatical equivalents thereof limit the scope of a claim to the specified materials or steps and those that do not materially affect the basic and novel characteristic or characteristics of the claimed invention.

While the invention has been described in detail and with reference to specific embodiments thereof, it will be apparent to one skilled in the art that various changes and modifications can be made without departing from the spirit and scope of the invention.

What is claimed is:

1. A pharmaceutical composition comprising: (a) a material selected from the group consisting of mast cell stabilizers, antihistamines, salts thereof, and combinations thereof; and (b) a vasoconstrictor or a salt thereof; provided when said material consists of an antihistamine and salts thereof, at least one antihistamine other than ketotifen or a salt thereof is present.

2. The composition of claim 1, wherein the composition has an initial pH of equal to or less than about 5.

3. The composition of claim 2, wherein said pH is in a range from about 4.3 to 5.

4. The composition of claim 1, wherein said antihistamine is selected from the group consisting of mepyramine, antazoline, diphenhydramine, carbinoxamine, doxylamine, clemastine, dimenhydrinate, pheniramine, chlorphenamine, dexchlorphenamine, brompheniramine, triprolidine, cyclizine, chlorcyclizine, hydroxyzine, meclizine, promethazine, alimemazine (trimeprazine), cyproheptadine, azatidine, acrivastine, astemizole, cetirizine, loratadine, mizolastine, azelastine, levocabastine, olopatadine, levocetirizine, desloratadine, fexofenadine, salts thereof, and combinations thereof.

5. The composition of claim 1, wherein said antihistamine is selected from the group consisting of acrivastine, astemizole, cetirizine, loratadine, mizolastine, azelastine, levocabastine, olopatadine, levocetirizine, desloratadine, fexofenadine, salts thereof, and combinations thereof.

6. The composition of claim 1, wherein said antihistamine comprises olopatadine or a salt thereof.

7. The composition of claim 1, wherein said mast cell stabilizer is selected from the group consisting of Cromolyn (Cromoglycate), Nedocromil, salts thereof, and combinations thereof.

8. The composition of claim 1, wherein said vasoconstrictor is selected from the group consisting of tetrahydrozoline, ephedrine, oxymetazoline, phenylephrine, pseudoephedrine, tramazoline, xylometazoline, naphazoline, salts thereof, and combinations thereof.

9. A pharmaceutical composition comprising: (a) olopatadine or a salt thereof in a concentration from about 0.001 to about 0.5% (w/v); and (b) naphazoline or a salt thereof in a concentration from about 0.001 to about 0.5% (w/v).

10. The composition of claim 9, further comprising ketotifen or a salt thereof in a concentration from about 0.001 to about 0.5% (w/v).

11. The composition of claim 9, wherein said olopatadine or salt thereof is present in a concentration in a range from about 0.01 to about 0.3% (w/v).

12. The composition of claim 10, wherein said ketotifen or a salt thereof in a concentration from about 0.01 to about 0.05% (w/v).

13. The composition of claim 9, wherein said composition has an initial pH in a range from about 4.3 to about 5.

14. The composition of claim 10, wherein said composition has an initial pH in a range from about 4.3 to about 5.

15. The composition of claim 2, wherein no more than about 10% of the mast cell stabilizers, antihistamines, or salts thereof is degraded at 40° C. and 20% RH for at least 10 days.

16. The composition of claim 2, further comprising an additive selected from the group consisting of tonicity-adjusting agents, preservatives, viscosity-modifying agents, and combinations thereof.

17. The composition of claim 16, wherein said tonicity-adjusting agent comprises urea, glycerol, sorbitol, mannitol, propylene glycol, lactose, dextrose, sodium chloride, sodium sulfate, or a combinations thereof.

18. The composition of claim 17, wherein the composition has an osmolality in a range from about 200 to about 700 mOsm/kg.

19. A method of preparing a pharmaceutical composition, the method comprising: (a) admixing a plurality of ingredients comprising: (1) a material selected from the group consisting of mast cell stabilizers, antihistamines, salts thereof, and combinations thereof; (2) a vasoconstrictor or a salt thereof; and (3) a carrier to form a mixture; and (b) adjusting a pH of said mixture to less than or equal to 5 with a pH adjusting material, thereby producing the composition; provided when said material consists of an antihistamine or a salt thereof, at least one antihistamine other than ketotifen or a salt thereof is present.

20. A method for treating, ameliorating, or reducing a severity of allergic conjunctivitis or symptoms thereof, the method comprising topically administering a sufficient amount of a pharmaceutical composition to an affected eye of a subject; wherein said composition comprises: (a) a material selected from the group consisting of mast cell stabilizers, antihistamines, salts thereof, and combinations thereof; and (b) a vasoconstrictor or a salt thereof; provided when said material consists of an antihistamine or a salt thereof, at least one antihistamine other than ketotifen or a salt thereof is present.

21. The method of claim 20, wherein said material comprises olopatadine or a salt thereof in a concentration from about 0.001 to about 0.5% (w/v).

22. The method of claim 21, wherein said material further comprises ketotifen or a salt thereof in a concentration from about 0.01 to about 0.05% (w/v).

23. The method of claim 21, wherein said vasoconstrictor comprises naphazoline or a salt thereof.

24. The method of claim 20, wherein the composition is administered by applying to the ocular surface in the form of eye drops, in one or more drops once per day, twice per day, or three or more times per day.
